(19) United States
(12) Patent Application Publication
(10) Pub. No.: US 2010/0094108 A1
(43) Pub. Date: Apr. 15, 2010

(54) SYSTEM FOR DETERMINING AND MONITORING DESATURATION INDICES AND INSTANTANEOUS RESPIRATORY RATE

(75) Inventors: Juan Luis Rojas Ojeda, Cadiz (ES); Antonio Leon Jimenez, Cadiz (ES); Luis Felipe Crespo Foix, Cadiz (ES); Nicole Gross, Cadiz (ES); Daniel Sanchez Morillo, Cadiz (ES)

Correspondence Address: FISH & RICHARDSON P.C., P.O BOX 1022, Minneapolis, MN, 55440-1022, US

(73) Assignee: Universidad de Cadiz, Cadiz (ES)

(21) Appl. No.: 12/445,054

(22) PCT Filed: Apr. 26, 2007
(86) PCT No.: PCT/ES2007/000253

§ 371 Date: Oct. 5, 2009

(30) Foreign Application Priority Data
Oct. 10, 2006 (ES) P200602577

(51) Int. Cl. A61B 5/1455

(52) U.S. Cl. 600/323

(57) ABSTRACT

Determining and monitoring desaturation indices and instantaneous respiratory rate, based on extracting components from the blood oxygen saturation (SpO₂) signal captured by an oximeter, obtaining and processing the data in the frequency domain in order to detect respiratory events and determine values such as respiratory rate and deviations therefrom (tachypnea/bradypnea) and desaturation indices.

Bioengineering applications in the field of medicine include monitoring and assisting with the diagnosis of respiratory disorders for its use in anesthesia, intensive care units and healthcare emergencies and assisting in the diagnosis of sleep apnea/hypopnea syndrome (SAHS).

### TECHNICAL FIELD

This invention pertains to extracting components by means of the frequency analysis of the signal captured exclusively by an oximeter, obtaining and processing information from blood oxygen saturation (SpO₂) data. It is therefore linked to the field of bioengineering, with applications in the field of medicine, allowing monitoring and assisting with the diagnosis of respiratory disorders, for its use in hospitals and in the home, and for assisting with the diagnosis of sleep apnea-hypopnea syndrome (SAHS).

### BACKGROUND

Oximetry is essentially based on the so-called Beer-Lambert law, which allows calculating the concentration of a substance in solution from its optical absorption at a certain wavelength. In the case of blood, there are two substances relevant to oxygenation, which are hemoglobin (Hb) and oxyhemoglobin (HbO₂). Since the deoxygenation of blood causes an increasing absorption in the red band and a decreasing absorption in the infrared band, oximeters therefore have two wavelengths: red and infrared wavelengths, which allow distinguishing oxygenated hemoglobin from reduced hemoglobin. There are different types of equipment on the market, the operation principle of which is always the same.

The approach to the diagnosis of sleep apnea-hypopnea syndrome (SAHS) by means of determining the oxygen desaturation event index, comparing the oxygen saturation (SpO₂) values with the baseline of the patient or with a theoretical baseline corresponding to healthy patients, is frequent.

The rigorous interpretation of nocturnal oximetry requires knowing the normal oxygen saturation values during sleep.

Based on these clinical criteria, the works aimed at automated diagnosis establish several criteria for detecting events and calculating desaturation indices. Those based on measuring SpO₂ may establish two types of essential criteria which in turn apply different calculation methods, obtaining different results. In the cases of severe SAHS, they do not differ substantially, but in mild and moderate cases they can lead to possible errors in the diagnosis. In relation to the algorithms used by the manufacturers of measurement instruments for determining desaturation indices, they are not provided to the user, therefore it is difficult to establish reliable comparative criteria.

Obtaining the DI (desaturation index) requires establishing a reference level with respect to which the events occurring are determined. Obtaining this reference level or baseline level has given rise to several methods which are considered in this study as a reference for obtaining events.

There are basically two strategies which have been established:
Methods based on the deviation with respect to a certain baseline level.
Methods based on the decrease or increase speed (flanks).

The first group includes those algorithms in which the baseline level is obtained as a statistical average of the SpO₂ values during the total recorded during the experiment [4]. The baseline level is obtained as an average of the signal during the complete period of the experiment, excluding artifacts and faults in the measurement. For the detection of respiratory events, this approach is based on searching for decreases below 4% of this baseline level. In other algorithms, the baseline level is a moving average of the values located in the 95^{th} percentile for one or several minutes before the treated instant. Therefore, before the calculation of the moving average, all the data of the treated period will have been subjected to a statistical filtering, eliminating those values less than that corresponding to the 95^{th} percentile. This ensures high baseline level values leading to good practical results in the discrimination of respiratory events [3,4]. As a result of the applications of the percentile approach to filtering, the desaturation periods in the patient are automatically excluded in the calculation of the baseline level, and the dynamics of the time evolution of the oximetry of the patient are furthermore conserved [2].

The second group takes into account that the apnea events can be completely located below the standard baseline level of 90% and that even when the baseline level is a statistical average for each patient, a considerable number of desaturations cannot be detected due to the fact that the SpO₂ recoveries do not reach the threshold marked by the baseline. This situation leads to error in the calculation of the DI.

Rauscher [4] proposes 2 new methods which do not require determining the baseline level, since it only considers level increases or decreases during a certain time interval, or in other words, it exclusively considers the average slope of the SpO₂ signal during defined time intervals. According to this approach, Rauscher obtains event rates by means of detecting the number of decreases greater than 4% during a time interval not less than 40 seconds, and by means of detecting the number of recoveries greater than 3% in a time interval of at least 10 seconds. This process justifies the difference in the up- and down-slopes, adducing that the restorations respond more quickly to the restoration of the respiratory activity.

However, these statistical assessment measurements of the baseline or baseline level of blood oxygen saturation can be affected by the following elements altering the measurement based on the time domain [1]:
Low blood circulation level
High HbCO fraction (e.g.: smokers or people in toxic environments)
Deoxyhemoglobin
Anemic hypoxemia

Respiratory rates (bradypneas, tachypneas and normal respiration) are calculated through oronasal flow cannulas, extensometric gauges, accelerometers and other indirect measurement devices.

### LITERATURE

[1] Technik in der Kardiologie. A. Bolz, W. Urbaszek.Springer, 2001.
[2] Eusebi Chiner, Jaime Signes-Costa, Juan Manuel Arriero, et al. Nocturnal oximetry for the diagnosis of the sleep apnoea hypopnoea syndrome: a method to reduce the number of polysomnographies? THORAX 1999;54:968-971.
[3] J. C. Vazquez, W. H. Tsai, W. W. Flemons, A. Masuda, R. Brant, E. Hajduk, W. A. Whitelaw, J. E. Remmers. Automated analysis of digital oximetry in the diagnosis of obstructive sleep apnea. THORAX, vol. 55, pp. 302-307, 2000.
[4] Rauscher, H., Popp, W., and Zwick, H. Computerized detection of respiratory events during sleep from rapid increases in oxyhemoglobin saturation. LUNG 1991, 169:335-342.

### SUMMARY

A method of determining and monitoring desaturation indices and instantaneous respiration rate is based on extracting frequency components from the signal captured by an oximeter, and obtaining information from vital respiratory physiological data, for assisting with the automated diagnosis of problems related to hypoxia in general and the sleep apnea-hypopnea syndrome in particular, independently of the baseline and absolute SpO₂ values.

The method allows:
Integration of the calculation of the DI (desaturation index) and of the respiratory rhythms of the patient (normal, bradypneas and tachypneas) in current oximetry equipment, independently of the baseline and absolute values of the patient.
Processing of the captured data, by means of a microprocessor-based system, to extract and display the mentioned physiological variables: Spectro-oximetry and Spectro-apneas.
Display of parameters resulting from the analysis of the previous variables: respiratory rhythm and desaturation index/hour.
The application at home by storing or transmitting the data for its interpretation by a specialist.

### BRIEF DESCRIPTION OF THE DRAWINGS

FIG. 1-a shows the different information processing steps leading to the generation of the information about the desaturation index.

FIG. 1-b shows the different information processing steps leading to the generation of the information about the respiratory indices.

FIG. 2 schematically depicts a system that provides the desaturation index and respiratory indices based on oximeter data.

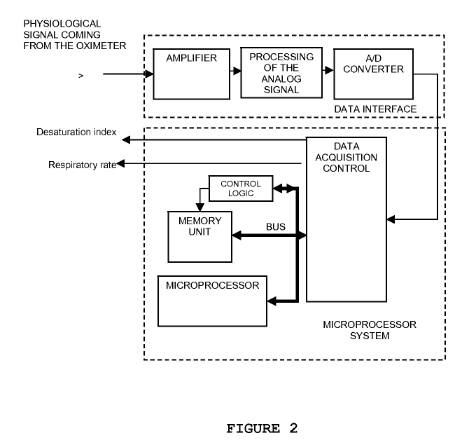

FIG. 3-a shows the form of the data captured by the oximeter.

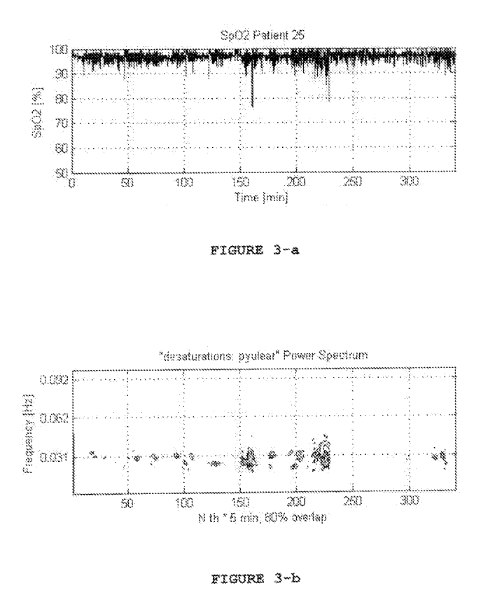

FIG. 3-b shows the corresponding spectral power of the oxygen desaturations (SpO₂) in the case of a patient with a diagnosis of medium desaturations (DI=25).

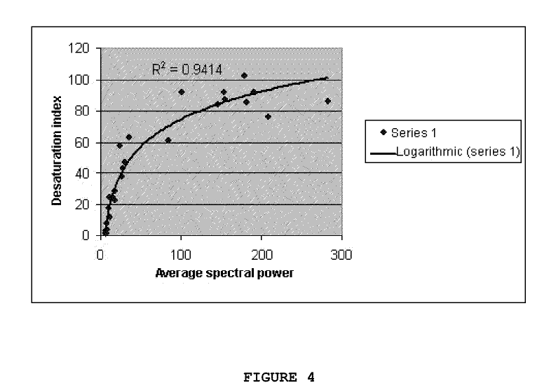

FIG. 4 shows the correlation existing between the calculated average spectral power in the ranges established for oxygen desaturations, and the corresponding desaturation indices established by the experts of the reference hospital.

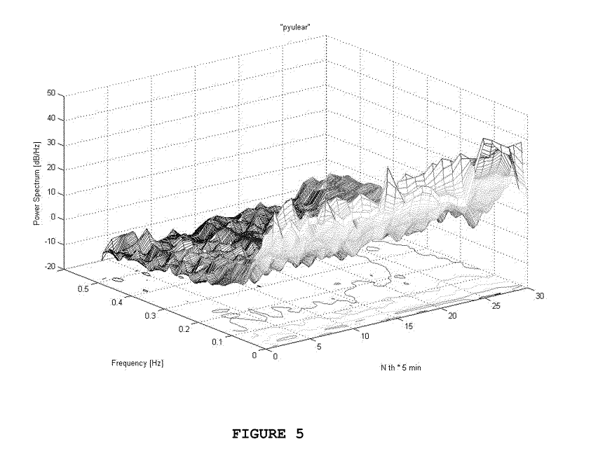

FIGS. 5 and 6 show the spectral location in the frequency ranges of the respiratory components and of the desaturations.

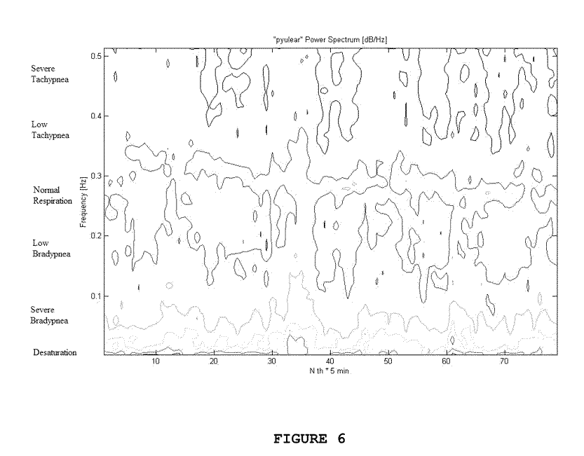

### DETAILED DESCRIPTION

A method is provided for calculating the desaturation index based on frequency analysis. Therefore, the method is not affected by the calculation of baselines used in traditional methods, or by the existence of artifacts outside or inside the patient which can modify the measurement. Likewise, a method which is not included in current oximeters is described for determining the respiratory rhythms of patients, also based on the frequency analysis of the respiratory rhythms detected in the signals provided by the oximeter.

The method described and set forth consequently involves a simplification of the tests for the diagnosis of certain dysfunctions associated with respiratory disorders such as the obstructive sleep apnea-hypopnea syndrome (SAHS), providing an aid for the diagnosis of respiratory disorders and for evaluation in risk situations.

The following are emphasized among the advantages provided with respect to the current state of the art:
1. System with a simple application and operation.
2. It does require calibrating the measurement.
3. It does not require skilled personnel.
4. Use at home and use in hospital.
5. Use in disaster and emergency situations for the quick discrimination of the vital situation of the affected people.
6. Novel processing of the captured information for information useful for the diagnosis.

The oximetry sensor provides an electrical signal, proportional to arterial oxyhemoglobin saturation (SaO₂). This electrical signal is transmitted to a processing circuitry, which amplifies the signal, filters it and converts it into a digital signal. The filtering parameters prior to the A/D conversion are conditioned by the sampling frequency. This filtering can be implemented through hardware or software or by means of a combination of both. The resulting digital signal is delivered to a microprocessor system for its evaluation.

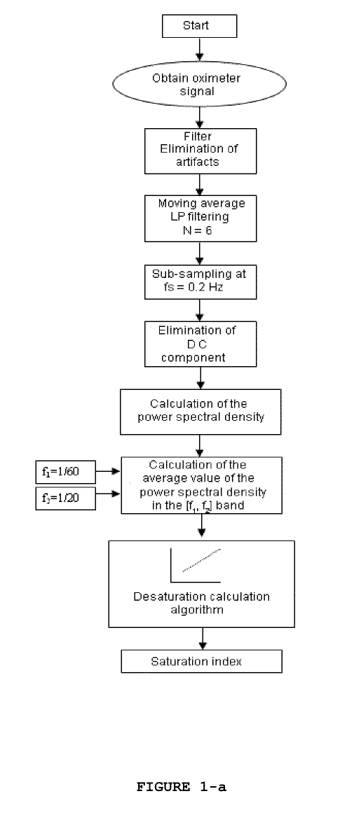

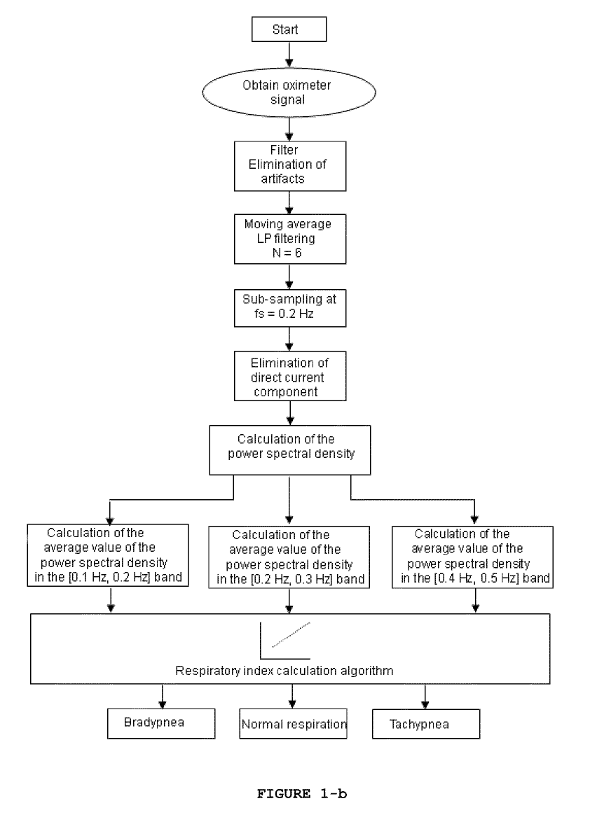

This system will operate according to instructions stored in memory, implementing the calculation process shown in FIGS. 1-a and 1-b. Furthermore, the system may store the data captured and obtained through the processing in a memory. The storage can be carried out in any storage system or combination thereof, such as volatile memories (DRAM), non-volatile memories, hard drives, CD-RW, DVD, removable memories (SD, MMC cards,and the like). The microprocessor system can furthermore display the results to the operator through a display, generate acoustic alerts, luminous alerts or alerts of any other type. It can contain input devices such as touch screens, keyboards, or any other device intended for the input of information by the operator.

It is evident that this microprocessor system can be physically implemented by one or several devices, capable of fulfilling the described functions. They can be general or specific purpose systems such as microprocessors, microcontrollers, digital signal processors, application-specific integrated circuits (ASICs), personal computers, PDAs, smartphones, and the like.

It is important to emphasize that the processing for obtaining the respiratory and desaturation indices is physically decoupled and can be made independent. In contrast, the preprocessing steps for the signal captured by the oximeter are identical. The collected signal is subjected to an initial filtering to eliminate artifacts in the measurement. A low-pass filtering is subsequently applied by a moving average filter, with a sample index for the average which can vary about 5 samples. The output of this filter is subjected to a sub-sampling to generate a cluster of samples at fs=0.2 Hz, from which the DC component is eliminated. The pre-processing block thus ends.

Both for obtaining the respiratory indices and for calculating the desaturation index, the power spectral density of the signal resulting from the previous preprocessing is calculated, using to that end any of the methods described in the literature (parametric or non-parametric).

For the calculation of the desaturation index, the average value of the previous spectral estimation in the [ 1/60 Hz, 1/20 Hz] band is calculated. This average value allows directly obtaining the DI value through the logarithmic ratio statistically linking both amounts, according to the adjustment performed with a control group. The desaturation index is stored by the system.

For the calculation of the respiratory indices, the average value of the spectral estimation in the [0.1 Hz, 0.2 Hz], [0.2 Hz, 0.3 Hz], [0.4 Hz, 0.5 Hz] bands is calculated. These average values again allow directly obtaining the normal respiration, bradypnea and tachypnea index values through the ratio statistically linking the respective amounts, according to the adjustment performed with a control group. The normal respiration, bradypnea and tachypnea indices are stored by the system.

In one aspect, the method includes the following phases:
1. Test for collecting data from the patient, with the placement of the oximetry sensor.
2. The acquisition system conditions the signal by means of a preamplifier amplifier and anti-aliasing filter. The sampling is done with frequencies not less than 1 Hz. The obtained data is stored in a record for its processing.
3. A prior filtering of the previous work space is applied to eliminate artifacts in the measurement, generating a new fault-free data record. This preprocessing can include the truncation or the interpolation on the original record.
4. A moving average (LP) filtering is applied, followed by a sub-sampling at a new rate of 0.2 Hz. The DC component is eliminated from the resulting signal.
5. Processing of the signal, to extract the desaturation index. The power spectral density is calculated and its average value in the [ 1/60, 1/20 Hz] band is evaluated.
6. Delivery of the result of the processing to a decision-making step, previously adjusted with a control group, to directly obtain the desaturation index from the previous average spectral value.
7. The desaturation index is determined immediately and can be presented to the patient by his or her specialist doctor in real time or as soon as the test ends.
8. Processing of the signal, to extract the respiratory indices. The power spectral density is calculated and its average value in the [0.1, 0.2 Hz], [0.2, 0.3 Hz] and [0.4, 0.5 Hz] bands is evaluated.
9. Delivery of the result of the processing to a decision-making step, previously adjusted with a control group, to directly obtain the bradypnea index, the normal respiration index and the tachypnea index from the previous average spectral values.
10. The bradypnea index, the normal respiration index and the tachypnea index are determined immediately and can be presented to the patient by his or her specialist doctor in real time or as soon as the test ends.

1-7. (canceled)

8. A method for determining and monitoring physiological parameters comprising:
detecting absorption of electromagnetic radiation by hemoglobin molecules in arterial blood of a subject with an oximeter, wherein the oximeter produces an electrical signal related to a percentage of the hemoglobin molecules saturated with oxygen;
conditioning the electrical signal;
digitizing the electrical signal;
processing the digitized signal to yield a power spectral density;
averaging the power spectral density in a selected band or frequency range to yield an average value of the power spectral density;
assessing an average number of oxygen desaturations experienced by the subject per unit of time using the average value of the power spectral density; and
providing an index based on the average number of oxygen desaturations experienced by the subject per unit of time.

9. The method of claim 8, wherein the selected band comprises frequencies between 1/60 Hz and 1/20 Hz.

10. The method of claim 8, wherein the index is a desaturation index.

11. The method of claim 8, further comprising averaging the power spectral density in a second set of bands to yield at least a second average value of the power spectral density, and obtaining an index related to an incidence of abnormally fast, abnormally slow, or normal respiratory rate using the power spectral density in the second set of bands.

12. A method for determining and monitoring physiological parameters comprising:
detecting absorption of electromagnetic radiation by hemoglobin molecules in arterial blood of a subject with an oximeter, wherein the oximeter produces an electrical signal related to a percentage of the hemoglobin molecules saturated with oxygen;
conditioning the electrical signal;
digitizing the electrical signal;
processing the digitized signal to yield a power spectral density;
averaging the power spectral density in a selected band to yield an average value of the power spectral density in the selected band; and
providing an index related to an incidence of abnormally fast, abnormally slow, or normal respiratory rate to facilitate diagnosis of respiratory function, wherein the index is related to the average value of the power spectral density in the selected band.

13. The method of claim 12, wherein the selected band comprises frequencies between 0.1 Hz and 0.2 Hz, and the index is indicative of the bradypnea index, the normal respiration index and the tachypnea index.

14. The method of claim 12, wherein the selected band comprises frequencies between 0.2 Hz and 0.3 Hz, and the index is indicative of the bradypnea index, the normal respiration index and the tachypnea index.

15. The method of claim 12, wherein the selected band comprises frequencies between 0.4 Hz and 0.5 Hz, and the index is indicative of the bradypnea index, the normal respiration index and the tachypnea index.

16. A respiratory diagnostic device comprising:
a data interface configured to receive an electrical signal from an oximetry sensor coupled to a subject and to digitize the electrical signal, wherein the electrical signal is related to blood oxygen saturation of the subject; and
a microprocessor system configured to receive the digitized signal from the data interface and to process the digitized signal to assess a blood oxygen saturation and a respiratory rate of the subject, based on a frequency analysis of the digitized signal.

17. The device of claim 16, wherein the microprocessor is further configured to process the digitized signal to yield a power spectral density.

18. The device of claim 17, wherein the microprocessor is further configured to average the power spectral density over a selected band to yield an average value of the power spectral density.

19. The device of claim 18, wherein the microprocessor is further configured to provide a desaturation index from the average value of the power spectral density over the selected band.

20. The device of claim 19, wherein the selected band comprises 1/60 Hz to 1/20 Hz.

21. The device of claim 18, wherein the microprocessor is further configured to provide a respiratory rate index from the average value of the power spectral density over the selected band.

22. The device of claim 21, wherein the selected band comprises 0.1 Hz to 0.2 Hz, and the respiratory rate index is indicative of bradypnea, normal respiration rate, tachypnea, or any combination thereof.

23. The device of claim 21, wherein the selected band comprises 0.2 Hz to 0.3 Hz, and the respiratory rate index is indicative of bradypnea, normal respiration rate, tachypnea, or any combination thereof

24. The device of claim 21, wherein the selected band comprises 0.4 Hz to 0.5 Hz, and the respiratory rate index indicative of bradypnea, normal respiration rate, tachypnea, or any combination thereof.

25. The device of claim 17, wherein the microprocessor is configured to extract variables related to a desaturation index and a respiratory rate from the power spectral density, and to generate diagnostic output information based on the variables.

26. The device of claim 16, wherein the device is operable to facilitate diagnosis of respiratory disorders.
